(19) United States
(12) Patent Application Publication
(10) Pub. No.: US 2015/0078646 A1
(43) Pub. Date: Mar. 19, 2015

(54) METHOD AND DEVICE FOR DISPLAYING AN OBJECT WITH THE AID OF X-RAYS

(71) Applicant: SIEMENS AKTIENGESELLSCHAFT, Muenchen (DE)

(72) Inventor: FRANK DENNERLEIN, ECKENTAL (DE)

(21) Appl. No.: 14/490,913

(22) Filed: Sep. 19, 2014

(30) Foreign Application Priority Data
Sep. 19, 2013 (DE) 102013218821.8

(51) Int. Cl. A61B 6/00; A61B 6/03; G06T 11/00

(52) U.S. Cl. CPC A61B 6/5258; A61B 6/502; G06T 11/008; A61B 6/032
USPC 382/131

(57) ABSTRACT

A method and a device for displaying an object with the aid of X-rays include recording a multiplicity of X-ray projections from which a volume data set characterizing the density of the object is reconstructed. Subsequently, density values along a ray are determined on the basis of the volume data set. By carrying out low pass filtering for the density values along the ray, and determining the maximum of the filtered density values (maxima of the filtered density values), values are obtained for displaying the object which simultaneously effectively image fine structures and are scarcely subject to the influence of artifacts.

### CROSS-REFERENCE TO RELATED APPLICATION

This application claims the priority, under 35 U.S.C. §119, of German Patent Application DE 10 2013 218 821.8, filed Sep. 19, 2013; the prior application is herewith incorporated by reference in its entirety.

### BACKGROUND OF THE INVENTION

### Field of the Invention

The invention relates to a method and a device for displaying an object with the aid of X-rays.

X-ray technology has become established as a standard method in medical diagnosis. It is based on the fact that X-rays transmitted through an object are attenuated in accordance with the absorption properties of the object. The X-rays transmitted through the object are recorded by a detector. Depending on the recording geometry, the recorded intensities constitute a measure which, in particular, delivers a statement concerning the density of the tissue penetrated by the X-rays.

Traditional X-ray technology typically delivers projection images in two dimensions which have been recorded by a flat-panel detector. However, a resolution perpendicular to the detector surface has not so far been possible. In the course of the development of X-ray technology, methods have been developed which also deliver information relating to the third dimension. The methods are based on the fact that radiographs are taken from a multiplicity of various directions, and that density values of the object in three dimensions (generally denoted as voxels) are obtained from the X-ray images thereby obtained. The voxels, which correspond to density values or so-called grayscale values at points in space, can be used to analyze the object, for example by calculating sections of the object and displaying them.

The first X-ray modality that facilitated the reconstruction of a volume data set was computed tomography, which permits the rotation of the X-ray source about the object and/or about the patient. Since then, there have been a range of other X-ray units which allow a three-dimensional reconstruction, for example C-arms and mammography units. In mammography, units for 3D reconstruction are constructed in such a way that it is possible to traverse an angular range and take radiographs for the angular range. The term tomosynthesis is applied in this context. In contrast to computed tomography, it is frequently impossible in the case of other applications (for example tomosynthesis) to undertake recordings from an arbitrarily large angular range, and this can result in artifacts (the latter also being denoted below as angular artifacts).

Particularly in mammography, specific challenges arise regarding the display of data sets obtained by tomosynthesis, the challenges resulting, on one hand, from the fact that only a limited angular range, and thus artifact-affected volume data are used and, on the other hand, from the fact that relevant structures to be displayed (so-called microcalcifications indicating cancerous tissue) have a very small size.

In addition to sectional displays, further techniques are used in displaying volume data sets, which are usually also denoted as volume rendering and which take into account the fact that the aim is to display a volume (that is to say a three-dimensional structure).

A first method for displaying volume data is the digital reconstruction of a radiograph (also denoted as digitally reconstructed radiograph (DRR)). This involves simulation or calculation of a two-dimensional radiograph from a three-dimensional volume data set of attenuation values, for example by integrating or summing up the volume data along viewing rays. Such methods are described, for example, in German Patent Application DE 10 2005 008 609 A1, corresponding to U.S. Pat. No. 7,653,226 and in German Patent Application DE 10 2012 200 661 A1.

In addition, another technique is customary, namely maximum intensity projection (MIP) as a method for image processing. In the course of maximum intensity projection, three-dimensional volume data sets or image data sets are converted into two-dimensional projection images by respectively selecting along the viewing direction (projection direction) the data point with the maximum intensity. One field of application is, for example, the display of CT angiography and magnetic resonance angiography data. In the data, the blood vessels generally have high signal intensities, and can therefore be effectively visualized by maximum intensity projection. Such a method is, for example, addressed in U.S. Patent Application Publication No. 2013/0064440 A1.

The two methods mentioned above have deficits which are also noticeable, in particular, in the field of mammo tomosynthesis (tomosynthesis in the field of mammography). Important structures with high contrast but of very small size, such as microcalcifications, for example, are frequently invisible in DRRs because they are lost, due to their small size, in the course of an averaging effect resulting when the DRRs are calculated. In contrast, MIPs typically retain small structures, but in this case are subject to image noise and are affected by artifacts that are to be ascribed to the small angular range and propagated into the projection from the volume data set.

This means that there is a need for a procedure that, in particular, permits the display of small structures and is comparatively robust in relation to impairments in the recording quality of the volume data set, such as noise and angular artifacts, for example.

### SUMMARY OF THE INVENTION

It is accordingly an object of the invention to provide a method and a device for displaying an object with the aid of X-rays, which overcome the hereinafore-mentioned disadvantages of the heretofore-known methods and devices of this general type and which provide such an improved procedure.

With the foregoing and other objects in view there is provided, in accordance with the invention, a method and a device for displaying an object with the aid of X-rays, in which a reconstruction of a volume data set characterizing the density of an examined object, typically from various recording angles, is undertaken on the basis of a multiplicity of X-ray projections. The volume data set is then used to determine density values along a ray (typically viewing ray). The determined values are subjected to low pass filtering, preferably by a convolution with the aid of a convolution core which is constructed for low pass filtering. In this case, it is firstly possible to determine all the density values, and then to undertake low pass filtering for each of the density values. However, it is also conceivable that directly after determination of a density value the low pass filtering is performed at once for the density value and the filtered value is then stored. The maximum is then determined for the filtered density values along the ray. If appropriate, a minimum can also be determined in this case instead of a maximum by appropriate reformulation of the mathematical problem. Such a reformulation is also to be included in the scope of protection of the claims, that is to say the term “maximum” is to be understood in the sense of “extreme” in the case of equivalent recastings of the mathematical problem.

Finally, the maximum of the filtered density values which is determined for the ray is used to display the object (for example on a monitor).

On one hand, the invention permits the positive sides of the conventional MIP method to be retained (correct display of small microcalcifications) and, at the same time, permits the disadvantages of MIP methods to be avoided (that is to say, in comparison to the MIP method the reduction of angular artifacts and image noise are ensured together with a better visibility of soft tissue).

Other features which are considered as characteristic for the invention are set forth in the appended claims.

Although the invention is illustrated and described herein as embodied in a method and a device for displaying an object with the aid of X-rays, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a front-elevational view of a mammography unit;

FIG. 2 is a schematic and block diagram illustrating a conventional tomosynthesis recording;

FIG. 3 is a flowchart of a method according to the present invention;

FIG. 4 is a schematic illustration of a projection geometry; and

FIG. 5 is a group of images provided for comparison of the image quality for various recording techniques.

### DETAILED DESCRIPTION OF THE INVENTION

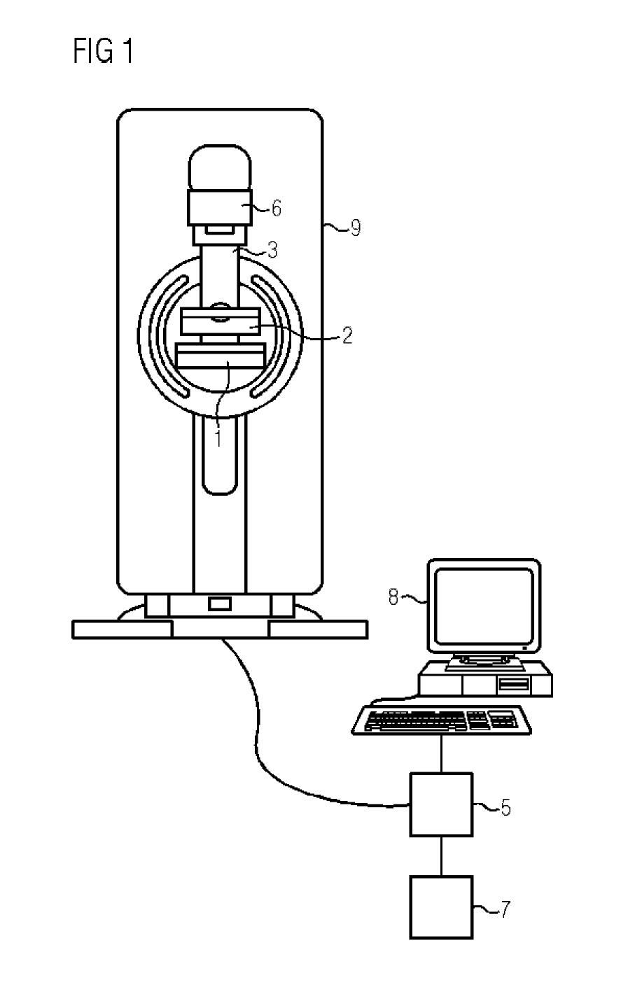

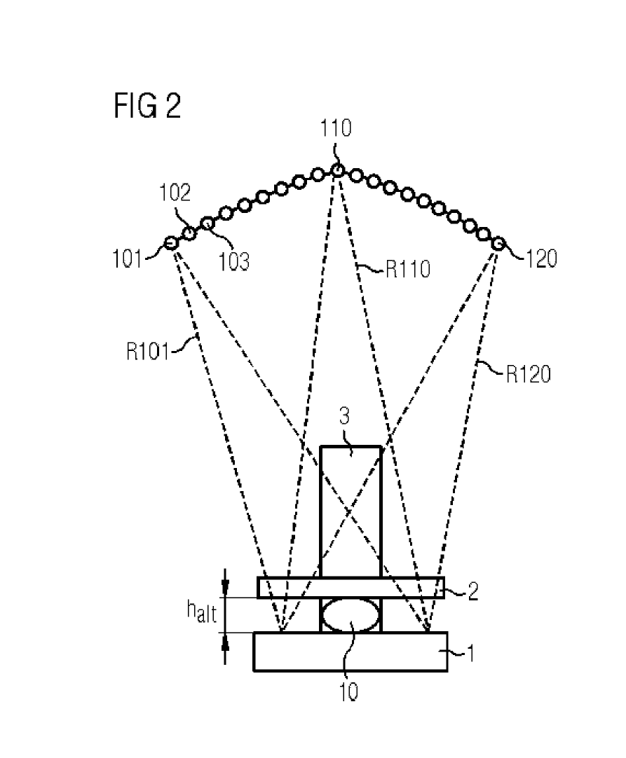

Referring now to the figures of the drawings in detail and first, particularly, to FIG. 1 thereof, there is seen a front view of a mammography unit 9. An object table 1 (usually including a detector) and a compression plate 2 used to compress a breast 10 to be examined (see FIG. 2), are disposed on a holder 3. In order to provide tomosynthesis recordings, an emitter 6 is embodied to be able to rotate about a rotation axis perpendicular to the plane of the drawing. Recorded projections can be fed to an evaluation computer 5. The evaluation computer is used, for example, for image reconstruction. In addition, in order to display calculated images the computer is normally connected to a display unit or a monitor 8 and frequently also to a memory unit 7 in which, for example, so-called filters, that is to say auxiliary variables for the calculation, and similar variables, can be stored.

The general situation for tomosynthesis recordings is illustrated in FIG. 2. The compression plate 2 has a wider construction than for conventional recordings, because of the recordings from various angular positions (typically minus 25° to plus 25°). The X-ray source or the emitter (indicated by reference numeral 6 in FIG. 1) traverses a trajectory during a tomosynthesis recording of an object 10. Positions 101, 102, 103 . . . for which a radiograph is taken in each case are marked on the trajectory. By way of example, the positions reproduce the locus of the focus of the X-ray source for the recordings. An expanding X-ray beam is illustrated for three positions 101, 110 and 120. The shape of the X-ray beam is that of a fan or a cone in most cases.

A volume data set is reconstructed from the recorded projections. Customary reconstruction methods are filtered back projection (FBP) and iterative methods (for example Feldkamp algorithm). The volume data set is usually present in the form of voxels, that is to say as density values (frequently denoted as grayscale values) which are assigned to points in space. For the analysis (at least) an imaging of the density values in space takes place onto values (frequently denoted as pixels) defined in two dimensions and used for display on a monitor. It is typical to proceed from viewing rays in this case. A pixel for display on a monitor is determined from the values of the volume data set along a viewing ray.

A modified MIP technique is proposed for this in accordance with the invention. The technique can also be denoted as a mollified maximum intensity projection (mMIP).

In this case, a mollified maximum intensity projection is carried out by a one-dimensional convolution-based low pass filtering of three-dimensional volume data along a virtual projection ray and by determining the maximum low-pass-filtered volume data along each projection ray in order to obtain the targeted projection value.

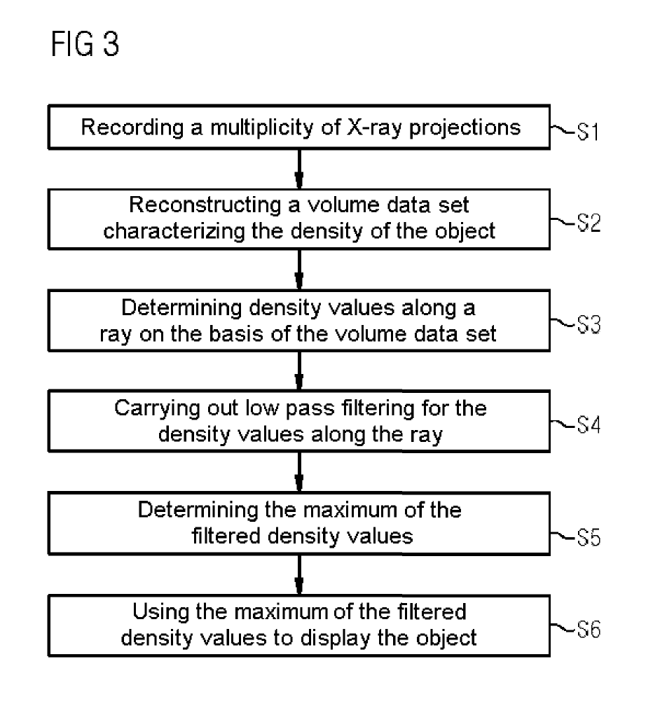

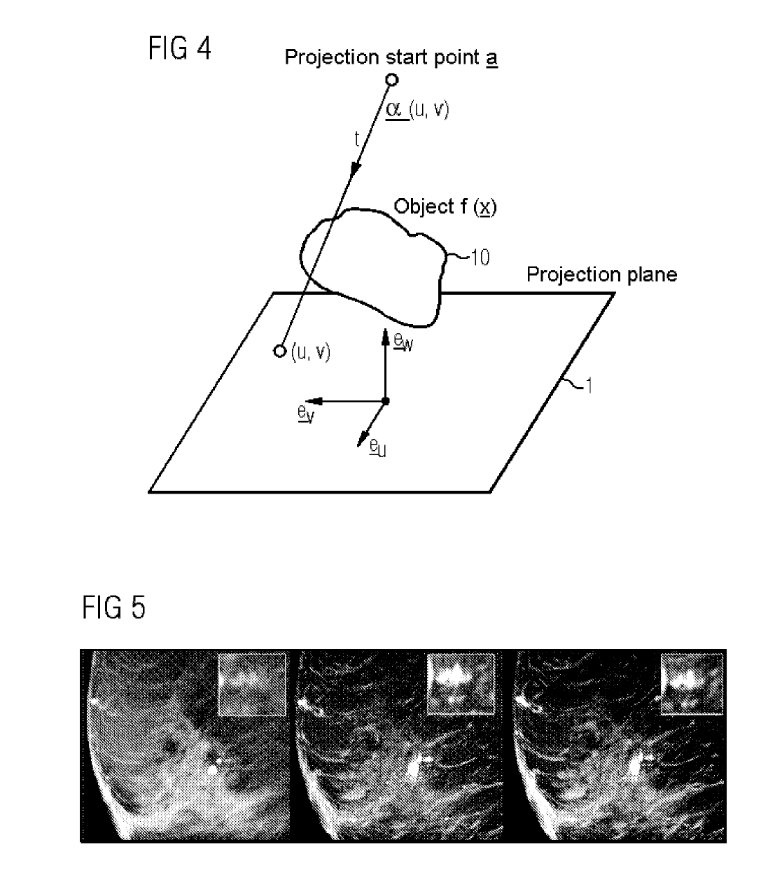

The method is illustrated in FIG. 3. Firstly, a multiplicity of X-ray projections is recorded (step S1) and a volume data set is reconstructed therefrom (step S2). Let f(x) denote the spatial distribution of the non-negative, reconstructed density of an object in the three-dimensional imaging space, in which x=(x,y,z) denotes a point in space. The targeted projection image (two-dimensional image formed of pixels intended for display on a monitor) to be obtained from the image data set or volume data set is denoted as g(u,v). In this case, u and v are the Cartesian coordinates, which denote pixel positions or positions in the projection image. As FIG. 4 shows, a conical ray projection geometry is assumed, and so the values g(u,v) can be determined from the values of f(x) along the ray which connects the point (u,v) and the projection center or the projection origin α. The ray is defined as α+tα(u,v), where t is a one-dimensional parameter, and the unit vector a defines the direction of the ray. The values from which g(u,v) is determined can then be denoted by v_{(u,v)}(t)=f(α+tα(u,v)). That is to say, the v_{(u,v)}(t) represent density values along the ray (step S3 in FIG. 3). Mollified MIPs g^{mMIP}(u,v) are defined as

g^{mMIP}(u,v)=ₜ^{max}(v_{(u,v)}(t) (1),

with the maximum being taken over the filtered values

v_{(u,v)}(t)=∫h(t−t)v_{(u,v)}(t)αt (2),

and h(t) denoting a one-dimensional convolution core. In this case, formula (2) corresponds to the low pass filtering in accordance with step S4, and formula (1) corresponds to the determination of the maximum in accordance with step S5 in FIG. 3.

The result of this is a mollified maximum intensity projection or a mollified MIP method obtained by carrying out a one-dimensional convolution-image-based filtering of the three-dimensional density f along virtual projection rays (the convolution corresponds to the imaging of the values v onto \tilde{v}), and by determining the maximum over the filtered \tilde{v}, which can be used to display the object (step S6 in FIG. 3). The mollified MIPs can be understood as a generalization of conventional MIPs and DRRs. A conventional MIP would be obtained by substituting the Dirac distribution (that is to say h(t)=δ(t) for the filter core. By contrast, a DRR would result from equating the filter core to the function h(t)=1. The mollified MIPs have the following properties:

A) They obtain the spatial resolution. For its calculation, the one-dimensional low pass filtering is always carried out along the rays projecting forward. This leads as a result to a lack of smearing between adjacent pixels in the two-dimensional projection image to be displayed. The spatial resolution is therefore not impaired.

B) The image noise is reduced in comparison to the conventional MIPs. The low pass filtering reduces high frequency noise in the values v along the projection ray. The subsequent search for the maximum will therefore be less likely to find individual values originating from noise, than to find a real object structure corresponding to the maximum density.

C) The contrast is heightened in comparison to DRRs. Small structures with high contrast are frequently lost in synthetically generated DRRs because of their small size. This does not apply to mollified MIPs, which are perfectly capable of visualizing small structures, since only the region at or around the structures is imaged in the projection image.

D) Angular artifacts are reduced in comparison to conventional MIPs. The artifacts are significantly suppressed by the application of a one-dimensional averaging operation over the artifact region. Low pass filtering leads to such an averaging, which is also responsible for the fact that angular artifacts (also termed limited angle artifacts, that is to say artifacts which are to be ascribed to the limited recording angular range) are suppressed in DRR projections.

FIG. 5 shows the projection calculated by using a DRR method (on the left), a conventional MIP method (in the middle) and a mollified MIP method (on the right). A small region, which includes microcalcifications, is respectively illustrated at the top right in a magnified zoom image. The DRR method only suggests the microcalcifications. The latter are more effectively reproduced in the conventional MIP method, while finally, the structure can be even more clearly discerned with the method according to the invention.

The present invention therefore permits volume data sets that have been obtained by using standard reconstruction methods (for example filtered back projection or iterative methods) to be obtained without the need for a special postprocessing of the data. Image noise and angular artifacts as well as out-of-plane artifacts are reduced in comparison to conventional MIP methods.

The invention has been illustrated above in the course of mammo tomosynthesis. However, the method is not limited to this application, but can be used in principle wherever volume data sets have been obtained by using X-rays.

1. A method for displaying an object with the aid of X-rays, the method comprising the following steps:
a) recording a multiplicity of X-ray projections;
b) reconstructing a volume data set characterizing a density of the object;
c) determining density values along a ray based on the volume data set;
d) carrying out low pass filtering for the density values along the ray;
e) determining a maximum of the filtered density values; and
f) displaying the object using the maximum of the filtered density values.

2. The method according to claim 1, which further comprises carrying out the low pass filtering step by convolution.

3. The method according to claim 1, which further comprises carrying out the method for a multiplicity of rays.

4. The method according to claim 1, which further comprises carrying out the steps of recording a multiplicity of X-ray projections and reconstructing a volume data set characterizing the density of the object during a tomosynthesis method.

5. A device for displaying an object with the aid of X-rays, the device comprising:
a) an X-ray unit configured to record a multiplicity of X-ray projections;
b) an arithmetic logic unit configured to: reconstruct a volume data set characterizing a density of the object, determine density values along a ray based on the volume data set, carry out low pass filtering for the density values along the ray, and determine a maximum of the filtered density values; and
c) a monitor configured to display the object using the maximum of the filtered density values.

6. The device according to claim 5, wherein said arithmetic logic unit is configured to carry out the low pass filtering by convolution.
